(19) United States
(12) Patent Application Publication
(10) Pub. No.: US 2024/0032821 A1
(43) Pub. Date: Feb. 1, 2024

(54) Thread-Based Strain Sensor and Data Processing for Quantifying Head Movement

(71) Applicant: Trustees of Tufts College, Medford, MA (US)

(72) Inventors: Sameer Sonkusale, Lincoln, MA (US); Yiwen Jiang, West Windsor, NJ (US); Eric Miller, Newton, MA (US)

(21) Appl. No.: 18/265,555

(22) PCT Filed: Dec. 6, 2021
(86) PCT No.: PCT/US2021/061968

§ 371 Date: Jun. 6, 2023

Related U.S. Application Data
(60) Provisional application No. 63/122,649, filed on Dec. 8, 2020.

(51) Int. Cl. A61B 5/11; A61B 5/00

(52) U.S. Cl. CPC A61B 5/1114; A61B 5/7267; A61B 5/6822; A61B 2560/0223

(57) ABSTRACT

An apparatus for sensing motion of a subject's head comprises three conductive threads, each of which has a resistance that varies in response to strain thereof, circuitry for applying a voltage across the threads, circuitry for detecting current flowing through the threads, a data-collection system configured to detect the current, and a data-processing system that classifies, based on data provided by the data-collection system, a type of motion of the head and an extent of the motion.

### RELATED APPLICATIONS

This application claims the benefit of the Dec. 8, 2020 priority date of U.S. Provisional Application 63/122,649, the contents of which are herein incorporated by reference.

### STATEMENT OF GOVERNMENT RIGHTS

This invention was made with government support under contract #1931978, which was awarded by the National Science Foundation. The government has certain rights in the invention.

### FIELD OF INVENTION

The invention relates to monitoring a subject and in particular to monitoring movement of a subject's anatomical features.

### BACKGROUND

In addition to communicating through spoken language, humans also use body language to communicate. When observing human body language, a particular portion of the body stands out above the others. That portion is the head.

Because of its abundance of sensory organs, observations of a subject's head, and in particular, its position and motion, are particularly useful for the study of human behavior. Information indicative of where the head is pointing is useful for understanding a subject's attention, focus and emotional state. Information indicative of how fast the head moved to arrive at its orientation is likewise indicative of the subject's state-of-mind.

In a classroom setting, head movements can be translated into students' gaze direction. As such, they can provide information on students' attentiveness during class. In the context of operating heavy machinery, observation of head motion provides a way to identify a distracted or tired operator. Head motions such as tilting and nodding when speaking or singing encode a person's emotional intent and serve as an indication for the speakers' interpersonal relationship in dialogs.

As a result of its importance, it is useful to accurately obtain information concerning the kinematic properties of a human's head.

Known methods of measuring the head's motion include the use of an inertial-measurement device to measure cervical range of motion. These methods are often used when attempting to diagnose neck pain. Such devices rely on the use of one or more of gyroscopes, accelerometers, and magnetometers. In general, two such sensors are used, with one being placed on the forehead and another on one of the vertebrae.

Other known methods of measuring motion include placement of a stretch band around the top of the subject's head and the use of virtual reality methods to measure the neck's range of motion.

In those cases where a less precise measurement is required, it is known to use radar to carry out such measurements, with either doppler radar or frequency-modulated continuous wave radar being used at wavelengths on the order of a millimeter. Also useful for less precise measurements is a machine vision system that carries out image processing on a live video stream to analyze head motion.

### SUMMARY

In one aspect, the invention features the use of thread-based sensors, or “sensory threads,” to monitor the head's motion and orientation in an efficient and flexible way. Embodiments include those in which a thread-based sensor comprises a material that directly measures strain and those that comprise a material having a property from which strain is inferred. Among these are embodiments in which an extrinsically applied coating of conductive material undergoes a change in its electrical conductivity as a result of strain.

Embodiments include those in which sensory threads are configured to interact with a measurement apparatus in connection with monitoring motion of other parts of the body, such as the various joints of a body. This provides a way to monitor movement of the various limbs and digits of an animal. Such information thus provides insight into movement associated with gait.

Embodiments also include those for collecting information resulting from motion that is not associated with movement about a joint. In one such embodiment, the apparatus incorporates sensory threads to capture the motion associated with inhalation and exhalation associated with respiration.

Embodiments also include those in which the apparatus relies on sensory threads for collection of data associated with perspiration or for gas detection or for detection of electromagnetic waves in a manner similar to that used by an antenna.

In one aspect, the invention features a method for monitoring and classifying motion of a subject's head using sensory threads by arranging two or more sensory threads so as to form an intersection. Among these methods are those in which first and second sensory threads are arranged so that the first thread crosses over the second thread and forms a thread intersection that has the general appearance of an “X,” a “+,” or an “L.” Other embodiments feature three or more threads that intersect to form an intersection having an appearance similar to that of an asterisk.

In a preferred embodiment, the sensory threads are carbon-coated threads that, when subjected to strain, undergo a change in some electrical property, such as conductivity. The sensory threads are coupled to an electronic interface for collecting data and providing that data to a data-processing device, thereby permitting the head's kinematic properties to be monitored in real time.

In some embodiments, data is collected through an impedance-readout board and communicated through a wireless interface to a data-processing device. The data is divided into fixed-length segments from which the data-processing device extracts a time series of features.

Based on the time series thus extracted, the data-processing device classifies the head's position as being in one of several different classes. Each class is the result of an associated rotation and an extent of that rotation. The number of classes for each rotation depends on the desired granularity. In a typical embodiment, there are nine classes: an equilibrium position, a small and large pitch of either sign, and a small and large yaw of either sign.

In some cases, the sensory threads are disposed on the nape of the subject's neck. This is useful since such a location is relatively unobtrusive and thus permits data collection to occur without hindering the subject's daily activities. However, other locations are available for placement of the sensory threads. These include lower portions of the back of the neck, the carotid triangle on either side of the neck, the submental triangle, and the posterior cervical triangle.

In one aspect, the invention features an apparatus for sensing motion of an anatomical feature of a subject. Such an apparatus includes three conductive threads, each of which has a resistance that varies in response to strain thereof, circuitry for applying a voltage across the threads, circuitry for detecting current flowing through the threads, a data-collection system configured to detect the current, and a data-processing system that classifies, based on data provided by the data-collection system, a type of motion of the head and an extent of the motion.

Among the embodiments are those in which the threads include first and second threads that intersect each other and that slopes that are equal in magnitude and opposite in sign. These threads are thus in a cruciform, or “X” shaped, configuration.

In other embodiments, the sensory threads are arranged to define a basis that spans the two-dimensional space defined by the surface of the subject's neck. The basis can but need not be an orthogonal basis. While it is convenient, for purposes of computation, for the sensory threads to also intersect, it is not necessary for this to be the case. In some embodiments, the number of sensory threads is greater than that required to define such a basis.

Also among the embodiments are those in which the data-collection system comprises a microcontroller board and electrodermal activity readout units corresponding to each of the threads. Examples of such units include impedance read-out boards.

In some embodiments, the anatomical feature comprises the subject's head. However, in others the anatomical feature is the subject's wrist, elbow, or knee.

Embodiments further include those in which the data-processing system is configured to classify the motion based on a correlation coefficient between data collected from different threads, those in which it is configured to classify the motion based on a likelihood to which the data indicates motion that exceeds a designated threshold, and those in which the data-processing system is configured to classify the motion based on a mean of a first difference for a data segment, the distance between the maximum and minimum point within a segment, and the correlation coefficient between data collected from the two sensors that are arranged in a cruciform configuration.

In another aspect, the invention provides an apparatus that includes a human-machine interface and a data-processing system in wireless communication with the human-machine interface. The human-machine interface relies on flexible strain-sensing threads that have been placed on a subject's neck to track head motion. The data-processing system receives data from the human-machine interface and executes a process for recognizing types of head motion and providing quantitative data representative of such motion in real-time by filtering and normalizing that data and dividing it into data segments. In some embodiments, a feature set extracted from each segment is provided as input to a support-vector-machine classifier for position prediction. In others, it is provided to a linear-support vector machine. In still others, it is provided to a kernel-naïve Bayes classifier.

In another aspect, the invention features an apparatus that includes sensory threads that are disposed to cross over at an intersection, data-collection circuitry that is in communication with the sensory threads, and a data-processing system that is in communication with the data-collection circuitry. Each of the sensory threads has an electrical property that varies in response to strain thereof. The data-collection circuitry receives, from each of the sensory threads, a signal indicative of that property. The data-processing system uses information derived from this signal to classify motion of the subject's head.

Embodiments include those in which the data-processing system includes a classifier that is trained by information provided by the data-collection system, those in which the data-processing system includes a support-vector machine, and those in which the data-processing system includes a classifier that relies on a Gaussian-kernel.

In some embodiments, the data-collection circuitry is configured to partition incoming signals from each of the threads into a series of overlapping segments.

Embodiments include those in which the data-processing circuitry is configured to classify motion of the subject's head during one of a series of overlapping segments that have been generated by partitioning incoming signals from each of the threads based on the segment's mean-value vector, those in which the data-processing circuitry is configured to classify motion of the subject's head during one of a series of overlapping segments that have been generated by partitioning incoming signals from each of the threads based on a difference between elements of the segment's mean-value vector, those in which the data-processing circuitry is configured to classify motion of the subject's head based on a resemblance between signals measured from different sensory threads, those in which the data-processing circuitry is configured to classify motion of the subject's head during a particular segment based on an intra-segment spread associated with the particular segment, the segment being one of a plurality of overlapping segments obtained by partitioning incoming signals from the sensory threads, those in which the data-processing circuitry is configured to classify motion of the subject's head based on correlation between signals measured from different sensory threads, and those in which the data-processing circuitry is configured to classify motion of the subject's head during a particular segment based on a difference between a minimum value measured during a segment that is one of a plurality of overlapping segments obtained by partitioning incoming signals from the sensory threads and a maximum value measured within the segment.

Also among the embodiments are those in which the sensory threads comprise elastic threads that have been coated with conductive ink and those in which the sensory threads comprise elastic threads that have been coated with ink that includes carbon, those in which each of the sensory threads is coated with a stretchable insulating material, and those in which each of the sensory threads is coated with silicone rubber.

Embodiments further include those in which the electrical property is conductivity, those in which it is resistivity, and those in which it is impedance.

Still other embodiments include those in which the data-collection circuitry includes a impedance read-out boards, each of which connects to one of the sensory threads and a microcontroller in communication with the impedance read-out boards. In such embodiments, the microcontroller is configured to filter and normalize signals from the impedance read-out boards and to provide overlapping segments of filtered and normalized data to the data-processing system for training the data-processing system to classify the motion of the subject's head.

In another aspect, the invention features placing sensory threads on a nape of a neck of a subject, the subject including a head that is coupled to the neck, connecting the sensory threads to a data-collection system, during movement of the subject's head, collecting, from each of the sensory threads, a signal indicative of motion of the subject's head, dividing the signals into overlapping data segments, providing the overlapping data segments to a data-processing system, using some of the overlapping data segments to train the data-processing system to classify motions of the head, and causing the data-processing system to use others of the data segments to classify motions of the head.

In some cases, the motion of the subject's head includes first and second excursions from an equilibrium position, the first excursions being in a first direction and the second excursions being in a second direction that is opposite the first direction. In such cases, practices of the foregoing method include normalizing the signals received from the threads. Such normalization includes using a first scale factor for normalizing the first excursions and a second scale factor, which differs from the first, for normalizing the second excursions.

In other practices, Z-score normalization is carried. In still others, each measurement is subtracted from the mean of the measurements and the resulting difference is divided by the standard deviation of the measurements.

In another aspect, the invention features manufacturing the sensory threads. Such a method includes, for each of the sensory threads, stretching the thread, applying conductive ink to the thread while the thread is stretched, releasing the thread, placing the thread in a hot oven, after having baked the thread, coating the thread in an insulating layer, and coupling connectors to ends of the thread.

An apparatus having the foregoing features promotes advances in physical rehabilitation and in augmented reality or virtual reality systems. In addition, such an interface is able to provide data that is useful for the study of human behavior.

All embodiments and practices described herein are non-abstract. Descriptions of abstract embodiments and practices have deliberately been omitted to avoid having the claims be misconstrued as covering abstract embodiments. Accordingly, the claims cover only non-abstract embodiments. As used herein, “non-abstract” means the converse of “abstract” as that term has been defined by the courts of the United States as of the filing of this application.

Any computers or data-processing systems that are encompassed by the claims are non-generic. Attempts to carry out the claimed subject matter using a generic computer have been unsuccessful. Accordingly, a non-generic computer was required.

Attempts were made to carry out the methods recited herein entirely within the human mind. All attempts were unsuccessful. Accordingly, it is believed that the methods described herein cannot practically be performed entirely in the human mind.

The claimed subject matter is integrated into a practical application, namely that of measuring head motion. This is practical for the same reason that the ability to measure other physical phenomena is practical.

### BRIEF DESCRIPTION OF THE FIGURES

FIG. 1 shows different orientations of a subject's head;

FIG. 2 shows first configuration of sensory threads on the nape of a subject's neck;

FIG. 3 shows a process for manufacturing a sensory thread as shown in FIG. 2;

FIG. 4 shows circuitry for inferring motion of the subject's head based on changes in the conductivity of the sensory threads shown in FIG. 3; and

FIG. 5 shows a data-processing method for processing data acquired by the circuitry shown in FIG. 4.

### DETAILED DESCRIPTION

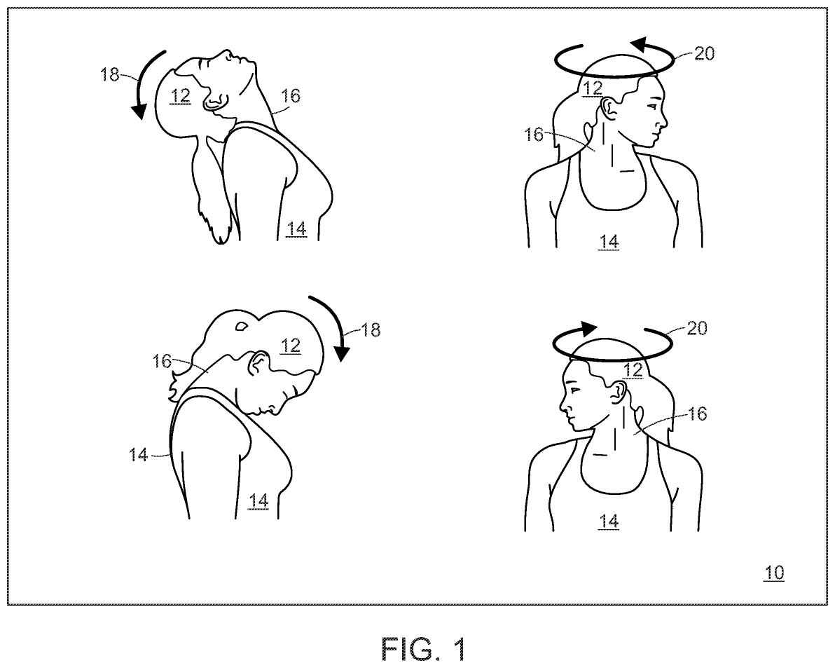

FIG. 1 shows four views of a subject 10 having a head 12 connected to a torso 14 by a neck 16. Each view shows the head 12 in a different orientation. Musculature within the neck 16 enables the subject 10 to move the head from one orientation into another. In particular, the musculature enables the subject 10 to vary the head's pitch 18 by some pitch angle and the head's yaw 20 by some yaw angle.

Two or more sensory threads 22, 24, 26 that form an intersection 28 on the nape 30 of the subject's neck 16 cooperate to provide a way to measure the pitch 18 and yaw 20 shown in FIG. 1. A suitable sensory thread 22, 24, 26 is an elastic thread that has been coated with an ink that comprises carbon. Such threads have conductivity that changes in response to strain. The sensory thread's variable conductivity in response to strain provides a way to measure activity by musculature in the neck 16 that occurs while re-orienting the head 12. Such measurements provide information from which the various kinematic properties of the head 12, for example, orientation, angular velocity, and angular acceleration, can be inferred.

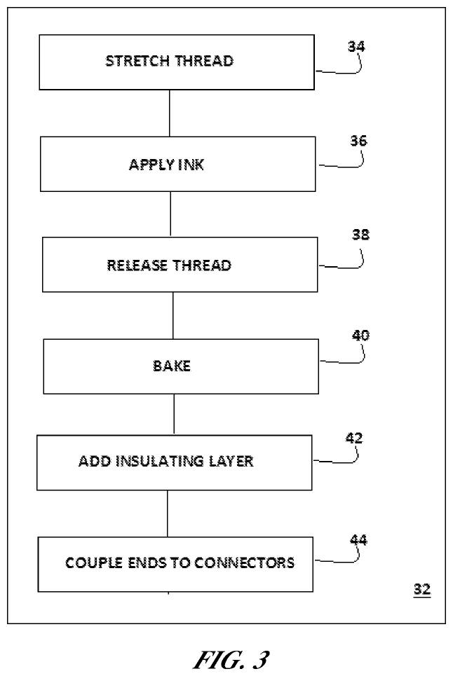

Referring now to FIG. 3, a process 32 for manufacturing a sensory thread 22, 24, 26 begins with providing an elastic thread. An example of such a thread is an elastic thread sold under the trade name “GÜTERMANN.” Such a thread is typically made form a combination polyester and polyurethane. A suitable ratio is 64% polyester and 36% polyurethane.

FIG. 3 shows a process 32 for manufacturing a suitable sensory thread 22, 24, 26. The process 32 begins with stretching the sensory thread to its maximum length (step 34) and coating it with a resistive ink (step 36). A suitable resistive ink is one that comprises carbon particles. Such an ink is sold under the tradename “C-200” by Applied Ink Solutions of Westborough, Massachusetts. The thread is then released so that it reverts to its equilibrium length (step 38).

During the coating step (step 36), it is important to ensure that every portion of the thread has been coated. In one practice of the manufacturing process 32, the thread is manually rubbed with the resistive ink to promote coating of every portion thereof. After having been coated in this manner and released into its relaxed state (step 38), the sensory thread 22, 24, 26 is baked in an oven at 80° C. for thirty minutes (step 40).

To reduce noise resulting from friction between the sensory thread 22, 24, 26 and the subject's skin, it is useful to add an insulating layer to the thread (step 42). A suitable insulating material from which such an insulating layer is manufactured is a silicone rubber. An example of such a material is platinum-catalyzed silicone sold under the tradename “ECOFLEX” by Smooth-On, Inc. of Macungie, Pennsylvania.

This insulating layer is added by holding one end of the sensory thread 22, 24, 26, dipping it into the insulating material, and hanging the now coated thread on a rack for several seconds to permit gravity evenly distribute the coating. The sensory thread 22, 24, 26, having thus been coated with insulating material, is then cured at room temperature. In a preferred practice, the process is repeated but with sensory thread 22, 24, 26 being held at its other end. The resulting two insulating layers provides greater resistance against abrasion.

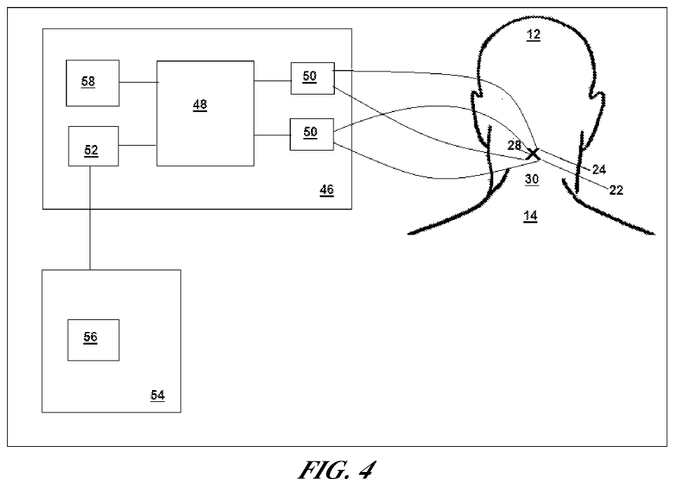

Each end of the sensory thread 22, 24, 26 is then coupled to connector (step 44). A suitable connector is a male metal crimp that is applied using a crimping tool. This provides a reliable wire connection that promotes the acquisition of a stable signal by circuitry 46 discussed in connection with FIG. 4.

The manufacturing procedure disclosed herein results in a sensory thread 22, 24, 26 having an inflexible central core with a fiber forming a helix that surrounds the core. Stretching the sensory thread 22, 24, 26 reorganizes the helical fiber by lengthening it, thus causing the core to compress. The act of stretching and coating (steps 34, 36) ensures that the carbon adheres to the thread's surface as well as to the fiber and to the core. This enables the sensory thread's resistance to increase upon stretching while reducing the incidence of surface cracks on the sensory thread's surface.

FIG. 4 shows circuitry 46 for sensing changes in the electrical properties of the sensory threads 22, 24 in response to strain caused by activation of musculature under the nape 30 of the subject's neck 16. The illustrated circuitry 46 processes collected data using a sliding window in a way that permits both complete motion identification and simultaneous motion classification.

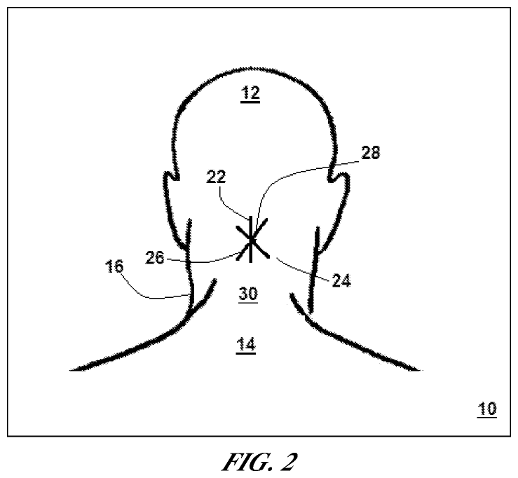

Unlike the configuration of sensory threads 22, 24, 26 shown in FIG. 2, that shown in FIG. 4 comprises only first and second sensory threads 22, 24. The first and second sensory threads 22, 24 that cross over to form a cruciform intersection 28 at a position that is estimated to be the middle of the region through which the cervical spine passes. However, it is not necessary to precisely locate the intersection 28. The sensory threads 22, 24 are fixed to the nape 30 by taping the two ends of the thread onto the skin. A suitable tape is a flexible adhesive tape such as that manufactured by 3M Corporation of Saint Paul, Minnesota under the name “NEXCARE.”

The illustrated circuitry 46 features a microcontroller 48. A suitable microcontroller 48 is that sold under the tradename “BITALINO.” However, other microcontrollers are also usable. It is particularly useful to provide a microcontroller 48 that is able to carry out real-time data streaming with a frequency of at least one kilohertz.

The microcontroller 48 receives input from as many impedance read-out boards 50 as there are sensory threads 22, 24. The connectors at each end of a sensory thread 22, 24 are soldered onto flexible thin wires, which are then soldered to input ports of each impedance read-out board 50.

A wireless interface 52 coupled to the microcontroller 48 permits data to be transmitted in real time to a nearby data processor 54 that is implements a classifier 56 that is to be trained using processed measurements provided by the microcontroller 48. A typical wireless interface 52 is a BLUETOOTH interface.

The circuitry 46 includes a power source 58. A typical power source 56 is a 4.7-volt lithium-ion polymer battery.

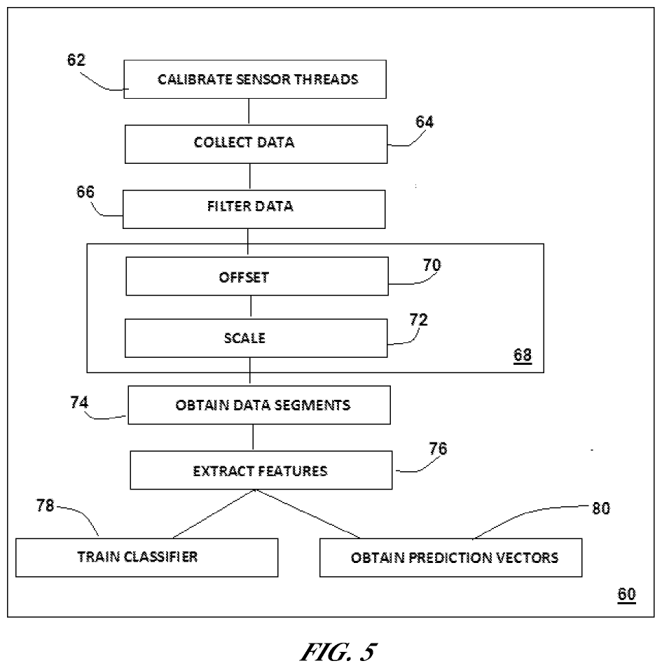

Although the manufacturing method shown in FIG. 3 reduces the incidence of cracking, it is difficult to completely eliminate surface cracks. Thus, a data-collection process 60, shown in FIG. 5, begins with calibrating the sensory threads 22, 24 to reduce performance deviations that arise between sensory threads 22, 24 as a result of different patterns of surface cracks (step 62). These different patterns often result in different sensory threads 22, 24 displaying different impedances for the same elongation. Additionally, since it is only the ends of the sensory threads 22, 24 that are taped onto the nape 30, a sensory thread's middle region is subject to spontaneous motions arising from causes other than head movement. A risk therefore arises that such motions will affect the sensory thread's electrical properties, including its impedance.

Such calibration typically includes making repetitive movements of a sensory thread 22, 24 taking measurements, and correlating measurements with movements to assess dependency of conductivity on movement. A typical calibration process begins by determining an equilibrium impedance associated with having the subject's head be at an equilibrium position, which is typically a front-facing position. The process continues with determining maximum and minimum impedance values of impedance over the head's full range of yaw angles and pitch angles. This is carried out by having the subject's head pitch or yaw to the maximum possible angle and return to the equilibrium position. This exercise is carried out several times.

Once the sensory threads 22, 24 have been suitably calibrated, data is collected using the circuitry 46 shown in FIG. 4 (step 64).

The collected data is then filtered to remove noise (step 66). This is carried out using a moving average filter. In a typical embodiment, the filter has a length of 120 samples and a sampling rate of one kilohertz. The filter thus acts as a low-pass filter having a −3 dB cut-off frequency at 147 Hz.

The filtered data is then normalized (step 68). Normalizing reduces the effects of manufacturing variations between sensory threads 22, 24. It also compensates for an asymmetry in the rate at which impedance changes as the sensory thread 22, 24 elongates and the rate at which it changes as the sensory thread 22, 24 relaxes.

Normalization (step 68) includes a translation step (step 70) and a scaling step (step 72).

The purpose of the translation step (step 70) is to pin a particular measured value to a known head orientation. In a preferred embodiment, a measured value of zero is pinned to the equilibrium orientation of the subject's head 12, namely that in which the subject 10 is facing forward. In such a case, translation includes adding an offset value to each measured data item such that the measured data value associated with a sensory thread 22, 24 that is in its relaxed and equilibrium position is zero. Positive and negative measurements are thus easily associated with elongation and relaxation of the sensory thread 22, 24.

The scaling step (step 72) scales the measured values so that they all lie in the interval [1, −1]. Because of the aforementioned asymmetry in the manner in which conductivity changes during stretching and relaxation, a first scale factor is used for positive values, i.e., values in [1, 0), and a second scale factor is used for negative values, i.e., values in (0, −1]. In a typical practice, positive values are divided by the maximum value, which was obtained during calibration, and negative values are divided by the minimum value, which was likewise obtained during calibration. This normalization process alleviates the effects of differences in the sensory threads' characteristics and eliminates the asymmetry between a stretching and relaxing sensory thread 22, 24.

The resulting data, after having been filtered and normalized, is divided into overlapping data segments (step 74). In a typical practice, a segment consists of 550 data points collected during the course of 0.55 seconds. This is followed by extraction of relevant features (step 76).

For each of the foregoing data segments, it is possible to identify a feature that is useful for serving as a basis for inferring position and motion of the subject's head 12.

A first feature is a segment's mean-value vector, the elements of which are mean values of measurements from each sensory thread 22, 24 during that segment.

A second feature is the difference between the two elements of the above-mentioned mean-value vector.

A third feature is a correlation coefficient that measures an extent to which measurements made using different sensory threads 22, 24 during a particular segment resemble each other.

A fourth feature is an intra-segment spread. A useful measure of intra-segment spread is the difference between a minimum value measured during a segment and a maximum value measured within that segment.

A fifth feature is a percentage of measured values that is within some pre-defined interval, with special cases being the percentage above or below some pre-defined threshold.

Other features include those that are derived by manipulating one or more of the foregoing features.

The selection of a suitable feature is guided to some extent by heuristic reasoning based on observations of features that have been observed in connection with particular types of motion. For example, if a first motion has been observed to result in first and second sensory threads 22, 24 outputting measured curves that are similar in shape a second motion has been observed to result in the same sensory threads 22, 24 outputting measured curves that are quite different in shape, then a heuristic rule would identify correlation as being useful for distinguishing the first and second motions from each other.

With one or more features having been selected, the process continues with training a classifier using a randomly selected fraction of available data segments (step 78). These randomly selected segments are used for training the classifier 56. The remaining segments are then used to obtain a prediction vector (step 80). The classifier's prediction vector is then checked for accuracy. In a preferred embodiment, the classifier 56 is a Gaussian-kernel support-vector machine and the fraction of segments used for training is 75%.

The data-processing method allows for two types of classification. The first type of classification includes identifying a completed motion from a time series. Examples of a completed motion include a completed pitch, either downward or upward, or a completed yaw, either clockwise or counterclockwise. This first type of classification uses, as features, both the correlation coefficient between measurements obtained from different sensory 22, 24 threads as well as the percentage of measured values that are above a designated threshold.

The second type of classification includes capturing motion from a sliding window. A leftward or rightward rotation is associated with a horizontal axis and an upward or downward flexing is associated with a vertical axis. The captured motion can thus be classified according to the location along either the horizontal or vertical axis. This second type of classification uses, as features, the mean, the mean of a first difference for a data segment, the distance between the maximum and minimum point within a segment, and the correlation coefficient between data collected from the two sensors that are arranged in the cruciform configuration.

The foregoing configuration results in easy and flexible detection of human motion in general. Although described primarily in connection with monitoring and classifying neck motion or head motion, the principles are agnostic to the moving structure. As such, the foregoing configuration is also adaptable for monitoring other bodily motions such as wrist motion, elbow motion, or knee motion. The use of a sliding window permits motion to be classified concurrently with data collection.

The configuration described herein is usable for a measurement apparatus that is both light in weight and simple to apply. These include consumer products that monitor one's posture, and in particular, the uprightness of one's position. The measurement apparatus is also useful for use in any research area in which collection of motion data is of importance, such as in psychological analysis. Moreover, since the configuration is economical and easy to produce, it has the potential to be used for monitoring motion of an ensemble of people, such as those in a large crowd.

Having described the invention, what is new and secured by Letters Patent is:

1. An apparatus for sensing motion of a subject's head, said apparatus comprising sensory threads disposed to cross over at an intersection, data-collection circuitry in communication with said sensory threads, and a data-processing system in communication with said data-collection circuitry, wherein each of said sensory threads has an electrical property that varies in response to strain thereof, wherein said data-collection circuitry receives, from each of said sensory threads, a signal indicative of said property, and wherein said data-processing system uses said signal to classify motion of said subject's head.

2. The apparatus of claim 1, wherein said data-processing system comprises a classifier that is trained by information provided by said data-collection system.

3. The apparatus of claim 1, wherein said data-processing system comprises a support-vector machine.

4. The apparatus of claim 1, wherein said data-processing system comprises a classifier that relies on a Gaussian-kernel.

5. The apparatus of claim 1, wherein said data-collection circuitry is configured to partition incoming signals from each of said threads into a series of overlapping segments.

6. The apparatus of claim 1, wherein said data-processing circuitry is configured to classify motion of said subject's head during one of a series of overlapping segments that have been generated by partitioning incoming signals from each of said threads based on said segment's mean-value vector.

7. The apparatus of claim 1, wherein said data-processing circuitry is configured to classify motion of said subject's head during one of a series of overlapping segments that have been generated by partitioning incoming signals from each of said threads based on a difference between elements of said segment's mean-value vector.

8. The apparatus of claim 1, wherein said data-processing circuitry is configured to classify motion of said subject's head based on a resemblance between signals measured from different sensory threads.

9. The apparatus of claim 1, wherein said data-processing circuitry is configured to classify motion of said subject's head during a particular segment based on an intra-segment spread associated with said particular segment, said segment being one of a plurality of overlapping segments obtained by partitioning incoming signals from said sensory threads.

10. The apparatus of claim 1, wherein said data-processing circuitry is configured to classify motion of said subject's head based on correlation between signals measured from different sensory threads.

11. The apparatus of claim 1, wherein said data-processing circuitry is configured to classify motion of said subject's head during a particular segment based on a difference between a minimum value measured during a segment that is one of a plurality of overlapping segments obtained by partitioning incoming signals from said sensory threads and a maximum value measured within said segment.

12. The apparatus of claim 1, wherein said sensory threads comprise elastic threads that have been coated with conductive ink.

13. The apparatus of claim 1, wherein said sensory threads comprise elastic threads that have been coated with ink that comprises carbon.

14. The apparatus of claim 1, wherein said electrical property is conductivity.

15. The apparatus of claim 1, wherein each of said sensory threads is coated with a stretchable insulating material.

16. The apparatus of claim 1, wherein each of said sensory threads is coated with silicone rubber.

17. The apparatus of claim 1, wherein said data-collection circuitry comprises a impedance read-out boards, each of which connects to one of said sensory threads and a microcontroller in communication with said impedance read-out boards, wherein said microcontroller is configured to filter and normalize signals from said impedance read-out boards and to provide overlapping segments of filtered and normalized data to said data-processing system for training said data-processing system to classify said motion of said subject's head.

18. A method comprising placing sensory threads on a neck of a subject, said subject comprising a head that is coupled to said neck, connecting said sensory threads to a data-collection system, during movement of said subject's head, collecting, from each of said sensory threads, a signal indicative of motion of said subject's head, dividing said signals into overlapping data segments, providing said overlapping data segments to a data-processing system, using some of said overlapping data segments to train said data-processing system to classify motions of said head, and causing said data-processing system to use others of said data segments to classify motions of said head.

19. The method of claim 18, wherein motion of said subject's head comprises first and second excursions from an equilibrium position, said first excursions being in a first direction and said second excursions being in a second direction that is opposite said first direction, said method further comprising normalizing said signals received from said threads, wherein normalizing said signals comprises using a first scale factor for normalizing said first excursions and a second scale factor for normalizing said second excursions, wherein said first scale factor is greater than said second scale factor.

20. The method of claim 18, further comprising manufacturing said sensory threads, wherein manufacturing said sensory threads comprises, for each of said sensory threads, stretching said thread, applying conductive ink to said thread while said thread is stretched, releasing said thread, placing said thread in a hot oven, after having baked said thread, coating said thread in an insulating layer, and coupling connectors to ends of said thread.

21. The method of claim 18, wherein placing said sensory threads on said neck comprises placing said sensory threads on the nape of said neck.

22. The method of claim 18, wherein placing sensory threads on said neck comprises placing said threads so as to form an intersection between said threads.

23. The method of claim 18, wherein placing sensory threads on said neck comprises placing said threads so as to form a basis that spans a two-dimensional space defined by said neck.

24. The method of claim 18, wherein placing sensory threads on said neck comprises placing three or more sensory threads on said neck.

25. The method of claim 18, wherein placing sensory threads on said neck comprises placing said threads on one of said subject's carotid triangles.

26. The method of claim 18, wherein placing sensory threads on said neck comprises placing said threads on said subject's cervical triangle.
